(19) United States
(12) Patent Application Publication
(10) Pub. No.: US 2023/0012541 A1
(43) Pub. Date: Jan. 19, 2023

(54) AUTHENTICATION DEVICE FOR USING THE BASE SEQUENCES OF DNA INCLUDED IN A MEDIUM

(71) Applicant: Young Taek PARK, Gochang-gun (KR)

(72) Inventor: Young Taek PARK, Gochang-gun (KR)

(21) Appl. No.: 17/827,712

(22) Filed: May 29, 2022

(30) Foreign Application Priority Data
Jul. 16, 2021 (KR) 10-2021-0093481

(51) Int. Cl. G16B 40/20; G16B 30/00

(52) U.S. Cl. CPC G16B 40/20; G16B 30/00

(57) ABSTRACT

The present disclosure relates to an authentication device using base sequence information of DNAs included in a medium, the authentication device including an authentication means provided in a medium, in which the plurality of DNA base sequence information are included in authentication information derived by reading out the authentication means composed of a plurality of DNAs.

### BACKGROUND OF THE INVENTION

### Field of the Invention

The present disclosure relates to an authentication device using base sequence information of DNA included in a medium, that is, an authentication device that authenticates a medium by comparing authentication information, which is DNA base sequence information derived by reading out an authentication means provided in a medium, with preset DNA base sequence information.

### Description of the Related Art

Recently, many problems are raised with whether off-line media are true. That is, many problems are generated with whether works of art such as a picture or a sculpture are true, whether documents are true, whether the origin of food being on the market is true, and whether other objects are true, and study on an authentication device for these items is being actively conducted.

Works of art are generally authenticated on the basis of special sensitivity of specialists of the fields of the works, and accordingly, uncertainty still remains in authentication.

Documents are generally authenticated by identifying handwriting in the documents, the materials of the documents, etc., and accordingly, authentication takes a long time. Further, such authentication is unavoidably secured by judgment of humans, so there is still a problem that authentication is unavoidably uncertain.

As for food, the demands by people for checking the places of origin of ingredients or whether there is genetic modification which can secure safety of food are greatly increasing, but the information that common customers can check about food is at the most the ingredients of food or the places of origin inscribed on the packs of food. Since there is a possibility of changing or hiding the places of origin, etc. inscribed on food by the providers of the food, there is a problem of low reliability.

In generally, barcodes, various certification marks, Void Tags, hologram stickers, etc. are used means for discriminating many products that are distributed or sold in the market or are used as means for authenticating places of origin. However, certification marks on products can be easily forged, and Void Tag or a hologram sticker can also be forged similar to the certification marks. In other words, barcodes, various certification marks, Void Tags, hologram stickers, etc. in the related art can be read or recognized only visually or only when a device is prepared in a visible area, and are fabricated through a common printing procedure, so there is a problem that they are easily forged/modified and several products can be easily copied.

As an alternative of such barcodes, various certification marks, Void Tags, hologram stickers, etc. which are easily forged/modified, recently, a Radio Frequency Identification (RFID) device employing an RFID technology is spotlighted. However, there is a problem that it is actually difficult to configure a system and achieve a result as expecting using the RFID technology in accordance with the features of products. For example, as a representative, there is a problem that it is difficult to authenticate stock farm products that were produced at specific places of origin and are distributed throughout the country, such as stock farm products grown at farms.

Accordingly, there is high necessity of developing an authentication device that can provide accurate determination about whether it is true in comparison to various authentication procedures in the related art.

### (Patent Document 1) KR 20-0372453 B

### SUMMARY OF THE INVENTION

In order to solve the problems of the related art described above, an object is to provide an authentication device that can secure accurate determination about whether a media is true.

In order to solve the problems according to the related art described above, an authentication device using DNA base sequence information included in a medium includes an authentication means provided in a medium, in which a plurality of DNA base sequence information may be included in authentication information derived by reading out the authentication means composed of a plurality of DNAs.

Preferably, the medium may be ink, inkpad, paper, mineral water, or instant noodles.

Preferably, the plurality of DNA base sequence information may include various kinds of DNA base sequence information having different base sequences of DNAs.

Preferably, the plurality of DNA base sequence information including the various kinds of DNA base sequence information may include a plurality of specific kinds of DNA base sequence information.

In a first ratio of the number of any one specific kind of DNA base sequence information to the number of all of the plurality of specific kinds of DNA base sequence information and a second ratio of the number of another one specific kind of DNA base sequence information to the number of all of the plurality of specific kinds of DNA base sequence information, the second ratio to the first ratio may be in a predetermined range.

Preferably, in a unit medium composed of the medium, the ratio of the number of any one specific kind of DNA base sequence information in authentication information derived by reading out an authentication means included in a portion of the unit medium to the ratio of the number of any one specific kind of DNA base sequence information in the authentication information derived by reading out an authentication means included in the entire unit medium may be in a predetermined range.

According to the subject of the present disclosure, any medium or object is authenticated using DNA base sequence information in an authentication procedure, so it is possible to secure accurate authentication about whether a medium is true on the basis of DNA base sequence information that cannot be easily copied.

In detail, any medium or object is authenticated using DNA base sequence information, and the ratio of the number of another one specific kind of DNA base sequence information to the ratio of the number of any one specific kind of DNA base sequence information is maintained to be constant in all of specific kinds of DNA base sequence information, so it is possible to authenticate any medium or object on the basis of authentication information including coincident DNA base sequence information, whereby it is possible to accurately determine whether it is true. Further, since whether it is true is accurately determined, it is possible to considerably decrease forged medium.

Further, according to the subject of the present disclosure, it is possible to secure accurate authentication of any off-line medium having an inherent recognition value stored in an on-line NFT.

### BRIEF DESCRIPTION OF THE DRAWINGS

The above and other objectives, features and other advantages of the present invention will be more clearly understood from the following detailed description when taken in conjunction with the accompanying drawings, in which:

FIG. 1 is a diagram conceptually showing an embodiment of the present disclosure; and

FIG. 2 is a diagram schematically showing DNA base sequence information included in authentication information in the present disclosure.

### DETAILED DESCRIPTION OF THE INVENTION

Hereinafter, preferred embodiments of a method according to the present disclosure will be described with reference to the accompanying drawings. The thicknesses of lines or sizes of components illustrated in the drawings may be exaggerated for the clarity and convenience of the following description. Further, the terminologies described below are terminologies determined in consideration of the functions in the present disclosure and may be construed in different ways by the intention of users or operators. Therefore, the definitions of the terminologies should be construed on the basis of the contents throughout the specification.

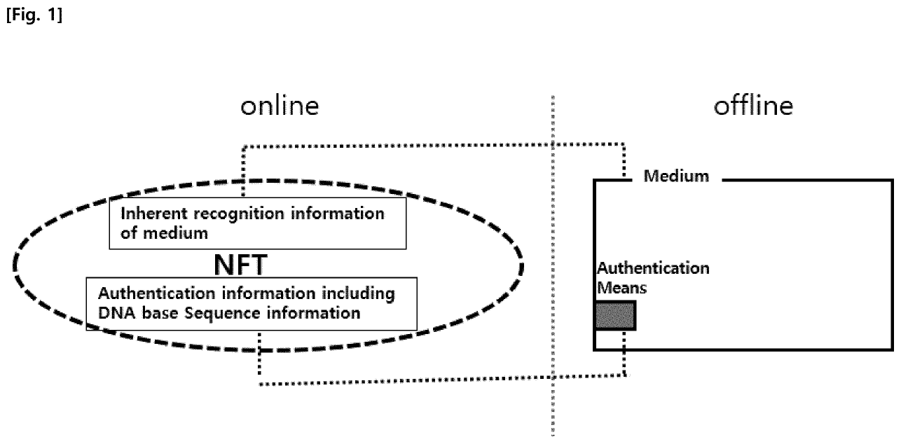

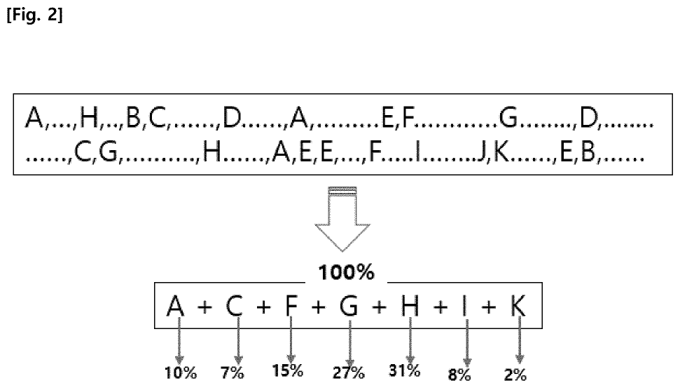

An embodiment of the present disclosure is described with reference to FIGS. 1 and 2.

First embodiment is described.

An authentication device according to the present disclosure may include an authentication means and a Non-fungible Token (NFT) provided in a medium. An inherent recognition value of the medium may be stored in the NFT.

First authentication information may be input in the NFT and second authentication information may be derived by reading out an authentication means composed of a plurality of DNAs. An authentication value authenticated as the first authentication information to an authentication value authenticated as the second authentication information may be in a predetermined range.

That is, when the authentication value authenticated as the second authentication information is an authentication value within a predetermined range from the authentication value authenticated as the first authentication information, the medium may be authenticated as a true medium corresponding to the inherent recognition value admitted through an NFT.

For example, when the authentication value authenticated as the first authentication information is 10% and the authentication value authenticated as the second authentication information is in the range of 6% to 8%, the medium may be authenticated as a true medium corresponding to an inherent recognition value admitted through an NFT. However, when the authentication value authenticated as the second authentication information is out of the range of 6% to 8% (is 4%, 9%, or the like), the medium cannot be authenticated as a true medium as a forged or copied medium that does not correspond to the inherent recognition value admitted through an NFT.

An NFT, which is a token that is not fungible, may be a token showing a digital asset having scarcity, and means a token that cannot be replaced with another token because it has an inherent recognition value as a token that is distributed on the basis of a block chain technology. That is, the NFT may be a means of which the ownership, a sale history, or the like can be clearly specified by converting existing property, such as a game, a work of art, a real asset, etc., into a digital token.

Inherent recognition values for works of art such as a picture, a sculpture, or the like in offline are included in an NFT, so ownership, a sale history, or the like of works of art can be clearly checked through the NFT. That is, inherent recognition values such as ownership, a sale history, or the like of works of art are created and stored in an NFT that is difficult to forge or copy, it is possible to accurately know ownership, etc. of works of art, etc.

However, even though a medium corresponding to an inherent recognition value stored in an NFT and an off-line medium authenticated through the NFT should be matched, there is a problem that when an NFT showing digital asset is transferred in online, the ownership of an actual medium such as a work of art should be transferred in offline, but it cannot be secured that a medium corresponding to a recognition value input in an NFT that is actually transferred in online is accurately transferred in offline.

Accordingly, it is required to make sure that a medium that is transferred in offline such as a work of art is a medium corresponding to an inherent recognition value in an NFT that is transferred in online.

According to a first embodiment of the present disclosure, an actual off-line medium may have an authentication means, second authentication information may be derived by reading out such an authentication means, and separate first authentication may be input in an NFT in which an inherent recognition value for the off-line actual medium is stored.

When an NFT is transferred in online, and accordingly, an actual medium is transferred with transfer of the NFT in offline, if an authentication value authenticated as the second authentication information to an authentication value authenticated as such first authentication information is in a predetermined range, the transferred medium can be authenticated as a medium corresponding to an inherent recognition value input in the transferred NFT. Authentication between an NFT in online and an medium in offline can be accurately made.

Technical characteristics of a plurality of DNA base sequence information included in authentication information are described with reference to FIG. 2.

Authentication information including the first authentication information and the second authentication information may include a plurality of DNA base sequence information, and the plurality of DNA base sequence information may be composed of various kinds of DNA base sequence information having different DNA base sequences. The DNA base sequence information may mean information that is discriminated by the base sequences of the DNAs.

In more detail, in a plurality of DNA base sequence information composed of various kinds of DNA base sequence information, the ratio of the number of another one specific kind of DNA base sequence information to the ratio of the number of any one specific kind of DNA base sequence information may be in a predetermined range, or preferably, the ratio of the number may be constant.

For example, as can be seen in FIG. 2, authentication information may include A, B, C, D, E, F, G, H, I, J, and K kinds and other kinds of DNA base sequence information. DNA base sequence information according to each kind is DNA base sequence information discriminated by the base sequence of different DNAs.

Authentication information according to the first embodiment of the present disclosure may be composed of a plurality of DNA base sequence information which are composed of various kinds of DNA base sequence information having different base sequences of DNAs. In these items of information, the ratio of the number of any one specific kind of DNA base sequence information in the entire may have a correlation within a predetermined range with the ratio of the number of another specific kind of DNA base sequence information in the entire. That is, the ratio of the number of another specific kind to the ratio of the number of any one specific kind may be in a predetermined range.

In FIG. 2 shown an example, the ratio of the number of DNA base sequence information of a kind A is 10% and the ratio of the number of DNA base sequence information of a kind D is 7%, so the ratio of the number of the kind C to the ratio of the number of the kind A may be 10:7.

Accordingly, in the first embodiment, the ratio of the number of the kind C to the ratio of the number of the kind A in a plurality of DNA base sequence information included in the first authentication information is 10:7. When the ratio of the number of the kind C to the ratio of the number of the kind A in a plurality of DNA base sequence information included in the second authentication information is 10:7, an authentication target medium may be authenticated as a medium corresponding to an inherent recognition value stored in a corresponding NFT. Further, even though it is 10:6 to 10:8 which is a predetermined range, an authentication target medium may be authenticated as a medium corresponding to an inherent recognition value stored in a corresponding NFT.

However, when it is out of the predetermined range, that is, when it is 10:7 in the first authentication information, but it is derived as being out of the range of 10:6 to 10:8 such as 10:9 or 10:5 in the second authentication information, the authentication target medium is not a medium corresponding to an inherent recognition value stored in a corresponding NFT, and accordingly, may be considered as being a forgery or a copy.

The example described above is only an example for explanation and does not limit the present disclosure.

The ratio of the numbers of specific kinds of DNA base sequence information within a predetermined range may be maintained to be limited to some limited specific kinds of DNA base sequence information.

That is, the plurality of DNA base sequence information including various kinds of DNA base sequence information may include a plurality of specific kinds of DNA base sequence information. It is possible to maintain the ratio of the numbers of specific kinds of DNA base sequence information while limiting the number of all of a plurality of specific kinds of DNA base sequence information to 100%.

In the example described above, A, C, F, G, H, I, and K kinds of DNA base sequence information are extracted from a plurality of DNA base sequence information, the ratio of the number of the A, C, F, G, H, I, and K kinds of DNA base sequence information is limited to 100%, and the DNA base sequence information of the kind A is set as 10% and the DNA base sequence information of the kind C is set as 6 to 8%, whereby it is possible to maintain the ratio of the number of another specific kind to the ratio of the number of any one specific kind within a predetermined range. The degree of authentication is further increased, so forgery or copy is impossible.

Further, the ratio of the number of any one specific kind of DNA base sequence information in authentication information derived by reading out the entire authentication means provided in an off-line medium and composed of a plurality DNAs may be in a predetermined range or preferably may be the same with respect to the ratio of any one specific kind of DNA base sequence information in authentication information derived by reading out only a portion of the authentication means.

That is, DNA base sequence information is not necessarily derived by reading out an entire authentication means composed of a plurality of DNAs, and even though DNA base sequence information is derived by reading out only a portion, similarly, the ratio of the numbers of specific kinds of DNA base sequence information may be maintained within a predetermined range.

In an entire authentication means provided in an off-line medium and composed of a plurality of DNAs, various kinds of DNAs having different base sequences may be uniformly distributed in the entire authentication means, so it is apparent that the ratio of the numbers of specific kinds of DNA base sequence information within a predetermined range may be maintained at any portions of the authentication means. Convenience of an authentication procedure can be increased with the degree of authentication increased.

Further, in a unit medium composed of a medium, it is apparent that the ratio of the number of any one specific kind of DNA base sequence information in authentication information derived by reading out an authentication means included in a portion of the unit medium to the ratio of the number of any one specific kind of DNA base sequence information in the authentication information derived by reading out an authentication means included in the entire unit medium may also be maintained within a predetermined range.

A plurality of DNAs distributed in an authentication means may be in a state in which a background DNA and a optionally produced DNA are mixed.

The background DNA may be a DNA existing in the natural world and can be obtained from animals or plants, and the optionally produced DNA may be a DNA optionally produced in an artificial method.

The DNA from which a specific kind of DNA base sequence information applied to the authentication procedure described above is derived may be such a DNA optionally produced in an artificial method.

That is, it may be a state in which a DNA existing in the natural world and a optionally produced DNA are mixed.

The base sequences of the optionally produced DNA are various kinds, and similarly, the base sequences of the background DNA are also various kinds, and the various kinds of base sequences of the optionally produced DNA may be different from the various kinds of base sequences of the background DNA.

Referring to FIG. 2 showing an example, as described above, A, C, F, G, H, I, and K kinds of DNAs are optionally produced DNAs and may be DNAs having a base sequence different from the base sequence of background DNAs of B, D, E, J, and other kinds. It is apparent that it is possible to check whether the base sequence of a optionally produced DNA and the base sequence of a background DNA are the same using a primer, etc. that are used for DNA amplification.

In order to use DNA base sequence information in an authentication procedure, it is possible to optionally produce a DNA in an artificial method and mix the DNA with a background DNA to be distributed in an authentication means in order to prepare a specific kind of DNA base sequence information that is found out in an authentication procedure process.

When it is determined that the base sequence of a background DNA is different from the base sequence of a optionally produced DNA, each optionally produced DNA can be produced by a necessary amount through amplification. Accordingly, it is apparent that it is possible to calculate the number of optionally produced DNAs in accordance with the weight and size of each optionally produced DNA known already while limiting the quantity of optionally produced DNAs that are put into all DNAs (background DNA+optionally produced DNA) distributed in an authentication means.

Accordingly, it is apparent that it is possible to calculate the ratio of numbers by comparing the numbers of specific kinds of DNA base sequence information derived by reading out optionally produced DNAs, that is, specific kinds of DNAs of all DNAs distributed in an authentication means.

Second embodiment is described.

In the second embodiment, an authentication means may be provided in a medium and a plurality of DNA base sequence information may be included in authentication information derived by reading out the authentication means composed of a plurality of DNAs.

In the second embodiment, the technical characteristics about a plurality of DNA base sequence information included in authentication information derived by reading out an authentication means composed of a plurality of DNAs follow the technical characteristics about a plurality of DNA base sequence information included in authentication information derived by reading out an authentication means composed of a plurality of DNAs in the first embodiment; and the technical characteristics about DNAs distributed in the authentication means also follow the technical characteristics about DNAs distributed in the authentication means described in the first embodiment described above.

It is apparent that the medium may be any medium as long as a plurality of DNAs can be uniformly distributed therein. Preferably, the medium may be ink, inkpad, paper, mineral water, or instant noodles.

When such medium are produced, a plurality of DNAs (background DNA+optionally produced DNA) having various kinds of base sequences are mixed in the medium, and the medium may be produced with the DNAs mixed to be able to be uniformly distributed throughout the medium.

A process in which an authentication means composed of a plurality of DNAs is mixed in a medium may be performed during the process of producing the medium or after the medium is completed in consideration of the properties of the medium or the production process of the medium.

For example, as for ink, it is apparent that it is possible to mix a plurality of DNAs during the process of producing the ink such that the DNAs are sufficiently stirred to be able to be uniformly distributed in the ink that is produced. DNAs that are mixed in ink of a unit amount to be produced are mixed in great amount and number (e.g., the number over one million composed of one million kinds of DNAs). When the DNAs are sufficiently stirred, the DNAs can be uniformly distributed in the portion of the ink of a unit amount. Accordingly, the ratio of the numbers of specific kinds of DNA base sequence information at any one portion may be the same as the ratio of the number of specific kinds of DNA at another portion or may be included in a predetermined range.

Since various kinds of a plurality of DNAs are mixed to be uniformly distributed, even in a plurality of DNA base sequence information derived by reading out an authentication means extracted not only from the entire unit medium composed of a medium, but also from a portion of the unit medium, similar to the first embodiment in which the technical characteristics of a plurality of DNA base sequence information, it is apparent that DNAs may be uniformly distributed such that the ratio of the number of another specific kind of DNA base sequence information to the ratio of the number of any one specific kind of DNA base sequence information can be maintained within a predetermined range, and it is apparent that the ratio of the numbers of specific kinds of DNA base sequence information can be maintained within some predetermined ranges limited in some limited specific kinds of DNA base sequence information.

In other words, the ratio of the number of a specific kind of DNA base sequence information derived by reading out an authentication means composed of a plurality of extracted DNAs or the ratio of the number of a specific kind of DNA base sequence information limited in some limited specific kinds of DNA base sequence information may be maintained to be constant even if they are derived by reading out an authentication means extracted from an entire medium or derived by reading out an authentication means extracted from a portion of a medium because a plurality of DNAs are uniformly mixed and distributed in the medium.

The authentication procedure may be made by determining whether the ratio of the number of a specific kind of DNA base sequence information, etc. corresponds to a predetermined range in a plurality of DNA base sequence information derived by reading out first an authentication means composed of a plurality of DNAs provided in a medium, similar to the first embodiment in which the technical characteristics about a plurality of DNA base sequence information are described.

Third embodiment is described.

In the third embodiment, a authentication means may be included and a plurality of DNA base sequence information may be included in authentication information derived by reading out the authentication means composed of a plurality of DNAs.

Unlike the first embodiment that is an authentication device performing an authentication procedure through an authentication means provided in a medium related to an NFT or the second embodiment that is an authentication device performing an authentication procedure through an authentication means provided in a medium, in the third embodiment, an authentication procedure may be performed by an authentication means itself composed of a plurality of DNAs in addition to the medium according to the first embodiment and the second embodiment.

As described above, an authentication procedure for whether a corresponding item is true may be performed not only when it is provided in the specific medium according the first embodiment and the second embodiment, but also when it is attached or provided to a product, a real property, a movable property, or an object such as an advertisement object requiring authentication.

For example, when an object is sold and it is required to authenticate the place of origin of the object, it is possible to determine whether the place of origin of the object is true from authentication information derived by reading out an authentication means attached and fixed to the object.

In the third embodiment, the technical characteristics about a plurality of DNA base sequence information included in authentication information derived by reading out an authentication means composed of a plurality of DNAs follow the technical characteristics about a plurality of DNA base sequence information included in authentication information derived by reading out an authentication means composed of a plurality of DNAs in the first embodiment; and the technical characteristics about DNAs distributed in the authentication means also follow the technical characteristics about DNAs distributed in the authentication means described in the first embodiment described above.

The authentication procedure may be made by determining whether the ratio of the number of a specific kind of DNA base sequence information, etc. corresponds to a predetermined range in a plurality of DNA base sequence information derived by reading out first an authentication means composed of a plurality of DNAs provided or attached to an object, etc., similar to the first embodiment in which the technical characteristics about a plurality of DNA base sequence information are described.

Although the present disclosure was described above so that those skilled in the art can easily understand and implement the present disclosure, but this is only an example and it would be understood that the present disclosure may be changed in various ways and implemented through equivalent examples by those skilled in the art. Accordingly, the protection range of the present disclosure should be determined by claims.

What is claimed is:

1. An authentication device comprising
an authentication means provided in a medium,
wherein a plurality of DNA base sequence information are included in authentication information derived by reading out the authentication means composed of a plurality of DNAs,
the medium is ink, inkpad, paper, mineral water, or instant noodles,
the plurality of DNAs constituting the authentication means include various kinds of DNAs obtained from the natural world and various kinds of DNAs artificially produced,
a DNA base sequence of the various kinds of DNAs obtained from the natural world is different from a DNA base sequence of the artificially produced various kinds of DNAs,
the number of the artificially produced various kinds of DNAs is calculated by reflecting the weight and size of each of the artificially produced various kinds of DNAs to quantity of the artificially produced various kinds of DNAs,
the plurality of DNA base sequence information include various kinds of DNA base sequence information having different base sequences of DNAs,
the plurality of DNA base sequence information including the various kinds of DNA base sequence information include a plurality of specific kinds of DNA base sequence information,
the plurality of specific kinds of DNA base sequence information are DNA base sequence information discriminated by DNA base sequences of the artificially produced various kinds of DNAs,
in a unit medium composed of the medium, the ratio of the number of any one specific kind of DNA base sequence information in authentication information derived by reading out an authentication means included in a portion of the unit medium to the ratio of the number of any one specific kind of DNA base sequence information in the authentication information derived by reading out an authentication means included in the entire unit medium is in a predetermined range,
the ratio of the number of the specific kinds of DNA base sequence information in the authentication information derived by reading out an authentication means included in another one portion of the entire unit medium to the ratio of the number of the specific kind of DNA base sequence information in the authentication information derived by reading out an authentication means included in any one portion of the entire unit medium is in a predetermined range, and
in a first ratio of the number of the any one specific kind of DNA base sequence information to the number of all of the plurality of specific kinds of DNA base sequence information in the authentication information derived by reading out an authentication means included in any one portion of the entire unit medium and a second ratio of the number of another one specific kind of DNA base sequence information to the number of all of the plurality of specific kinds of DNA base sequence information in the authentication information derived by reading out an authentication means included in any one portion of the entire unit medium, the second ratio to the first ratio is in a predetermined range.
